(19) United States
(12) Patent Application Publication
(10) Pub. No.: US 2024/0157306 A1
(43) Pub. Date: May 16, 2024

(54) HOLLOW-FIBER MEMBRANE AND METHOD MAKING THEREOF

(71) Applicant: 3M INNOVATIVE PROPERTIES COMPANY, St. Paul, MN (US)

(72) Inventors: Christian Krumm, Solingen (DE); Ramona Osterloh, Velbert (DE); Pierre-Alexandre Bourgeois, Essen (DE); Amy S. Determan, Mahtomedi, MN (US); Sven Frost, Essen (DE); Manfred Trussner, Radevormwald (DE); Mathias Stroschke, Sprockhövel (DE); Ki-Joo Sung, Minneapolis, MN (US)

(21) Appl. No.: 18/280,040

(22) PCT Filed: Mar. 10, 2022
(86) PCT No.: PCT/IB2022/052168

§ 371 Date: Sep. 1, 2023

Related U.S. Application Data
(60) Provisional application No. 63/264,398, filed on Nov. 22, 2021.
(60) Provisional application No. 63/167,754, filed on Mar. 30, 2021.

(51) Int. Cl. B01D 69/08; B01D 67/00; B01D 69/02; B01D 71/58; B01D 71/68; C12N 7/00

(52) U.S. Cl. CPC B01D 69/085; B01D 67/0016; B01D 69/02; B01D 69/088; B01D 71/58; B01D 71/68; C12N 7/00; B01D 2325/022; B01D 2325/02833; C12N 2750/14151

(57) ABSTRACT

A hollow-fiber membrane. The hollow-fiber membrane is made from a polymeric blend comprising an aromatic sulfone polymer and polyoxazoline; wherein the hollow-fiber membrane comprises an inner surface facing towards its lumen, an outer surface facing outwards and an intermediate wall having a wall thickness; wherein the hollow-fiber membrane is an integrally asymmetric, permeable hollow-fiber membrane.

### FIELD

The present disclosure relates to porous membranes. In addition, the present disclosure relates to a process for producing such membranes. The present disclosure further relates to use of such membranes for filtration and purification of liquid media.

### BACKGROUND

Hollow-fiber membranes are employed in a very wide range of different industrial, pharmaceutical or medical applications for precision filtration. In these applications, membrane separation processes are gaining in importance, as these processes offer the advantage that the substances to be separated are not thermally burdened or even damaged. Ultrafiltration membranes can be employed for the removal or separation of macromolecules. Numerous further applications of membrane separation processes are known from the beverages industry, biotechnology, water treatment or sewage technology. Such membranes are generally classified according to their retention capacity, i.e. according to their capacity for retaining particles or molecules of a certain size, or with respect to the size of the effective pores, i.e. the size of the pores that determine the separation behavior. Ultrafiltration membranes thereby cover the size range of the pores determining the separation behavior between roughly 0.01 and approx. 0.1 μm, so that particles or molecules with a size in the range larger than 20 000 or larger than approx. 200 000 Daltons can be retained. There is a need for better polymer membranes.

### SUMMARY

Thus, in one aspect, the present disclosure provides a hollow-fiber membrane; the hollow-fiber membrane made from a polymeric blend comprising an aromatic sulfone polymer and a polyoxazoline; wherein the hollow-fiber membrane comprises an inner surface facing towards its lumen, an outer surface facing outwards and an intermediate wall having a wall thickness; wherein the hollow-fiber membrane is an integrally asymmetric, permeable hollow-fiber membrane.

In another aspect, the present disclosure provides a method, the method comprising: providing a spinning solution comprising an aromatic sulfone polymer and a polyoxazoline, and a bore liquid comprising water, a solvent and a non-solvent; and spinning a hollow-fiber with a spinneret outer diameter for dope in the range of from 300 to 1000 μm, a spinneret needle outer diameter in the range of from 200 to 1000 μm and a spinneret needle inner diameter in the range of from 100 to 1000 μm.

Various aspects and advantages of exemplary embodiments of the present disclosure have been summarized The above Summary is not intended to describe each illustrated embodiment or every implementation of the present disclosure. Further features and advantages are disclosed in the embodiments that follow. The Drawings and the Detailed Description that follow more particularly exemplify certain embodiments using the principles disclosed herein.

### BRIEF DESCRIPTION OF THE DRAWINGS

FIG. 1 is a schematic perspective view, in partial cross-section of an exemplary hollow-fiber membrane.

FIG. 2 is a cross-section of an exemplary hollow-fiber membrane.

FIG. 3 is SEM picture of 4,000× magnification of cross-section of a hollow-fiber membrane according to the present disclosure.

FIG. 4 is SEM picture of 20,000× magnification of cross-section of a hollow-fiber membrane according to the present disclosure.

FIG. 5 is SEM picture of 4,000× magnification of cross-section of a hollow-fiber membrane according to the present disclosure.

FIG. 6 is SEM picture of 20,000× magnification of cross-section of a hollow-fiber membrane according to the present disclosure.

FIG. 7 is SEM picture of 4,000× magnification of cross-section of a hollow-fiber membrane according to the present disclosure.

FIG. 8 is SEM picture of 20,000× magnification of cross-section of a hollow-fiber membrane according to the present disclosure.

### DETAILED DESCRIPTION

Before any embodiments of the present disclosure are explained in detail, it is understood that the invention is not limited in its application to the details of use, construction, and the arrangement of components set forth in the following description. The invention is capable of other embodiments and of being practiced or of being carried out in various ways that will become apparent to a person of ordinary skill in the art upon reading the present disclosure. Also, it is understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. It is understood that other embodiments may be utilized and structural or logical changes may be made without departing from the scope of the present disclosure.

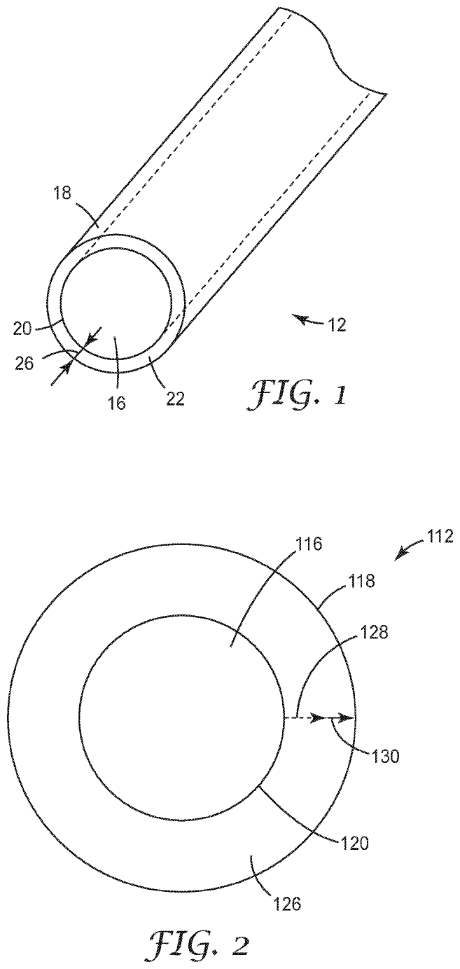

One embodiment of a hollow-fiber membrane according to the present disclosure is shown in FIG. 1. FIG. 1 illustrates a perspective view of a partial cross-section of a portion of an exemplary hollow-fiber membrane 12. Hollow-fiber membrane 12 may have a continuous hollow lumen 16, which extends from one end to the other end of the fiber, an outer surface 18 facing outwards, which forms an outer side of the fiber; an inner surface 20 facing towards the hollow lumen 16, which defines the limits of the continuous hollow lumen 16; and an intermediate wall 22 having a wall thickness 26. The hollow-fiber membrane 12 can be made from a polymeric blend comprising an aromatic sulfone polymer and a polyoxazoline. The hollow-fiber membrane can be an integrally asymmetric, permeable hollow-fiber membrane.

The wall thickness 26, measured between the outer surface 18 and the inner surface 20 of the hollow-fiber membrane 12, can be in the range of from 20 to 300 μm, from 30 to 200 μm, or from 40 to 80 μm.

Similarly, in order to achieve a desirable flow through the lumen of the hollow-fiber membranes according to the present disclosure, particularly, a favorable pressure drop, it is preferred that the inside diameter of the hollow-fiber membranes as described herein is in the range of from 50 to 800 μm, from 50 to 700 μm, from 50 to 600 μm, from 100 to 500 μm, from 100 to 400 μm, or from 100 to 300 μm. Wall thicknesses and diameters (i.e., inner or lumen diameter, and outer diameter) of the membranes as described herein are also determined by means of conventional examination methods, such as using scanning or transmission electron micrographs (SEM or TEM, respectively), for example with a magnification up to 20,000:1. In some embodiments, the hollow-fiber membrane can have tortuous structures extending from the inner surface toward to the outer surface. The inner upstream side of the membranes features a porous surface, which is build up by isotropic nodular structures. When pore compartments are connected in the membrane and therefore have torturous morphology in place, which leads to high trans membrane flow (TMF).

In some embodiments, the hollow-fiber membrane may have two zones: the zone with minimum pore size and the zone with maximum pore size. In some embodiments, the zone with minimum pore size adjoins the inner surface. In some of those embodiments, the zone with maximum pore size adjoins the outer surface. In other embodiments, the zone with minimum pore size adjoins the outer surface. In some of these embodiments, the zone with maximum pore size adjoins to the inner surface. “Adjoin” means that the zone of maximum or minimum pore size is located at a distance from the surface in the range between 0 to 8μm In some embodiments, the size of the pores in the zone with minimum pore sizes can be in the range of from 10 nm to 100 nm, from 10 nm to 90 nm, from 10 nm to 80 nm, from 10 nm to 70 nm, from 10 nm to 60 nm, from 20 nm to 80 nm, from 20 nm to 70 nm, from 20 nm to 60 nm, from 20 nm to 50 nm, from 30 nm to 70 nm, from 30 nm to 60 nm, from 30 nm to 50 nm, from 30 nm to 40 nm, from 40 nm to 90 nm, from 40 nm to 80 nm, from 40 nm to 70 nm, from 40 nm to 60 nm, or from 40 nm to 50 nm. In some embodiments, the size of the pores in the zone with minimum pore size can be less than 100 nm, 90 nm, 80 nm, 70 nm, 60 nm, 50 nm, 40 nm, 30 nm, or 20 nm. In some embodiments, the size of the pores in the zone with minimum pore size can be more than 10 nm, 15 nm, 20 nm, 25 nm, 30 nm, or 40 nm. In some embodiments, the size of the pores in the zone with maximum pore size can be in the range of from 0.05 μm to 10 μm. The average pore size of the zones with maximum pore size is larger than the average pore size of the zones with minimum pore size. The zone with minimum pore size can form a retention layer. When the zone with minimum pore size adjoins the outer surface, the retention layer adjoins the outer surface of the membrane and can form a more conducive membrane structure for filtering liquids, for example, biopharmaceuticals. In some embodiments the hollow-fiber membrane has a first zone of pores and a second zone of pores, wherein the first zone of pores adjoins the inner surface, and the second zone of pores adjoins the outer surface, and the density of pores in the first zone is greater than the density of pores in the second zone. In some embodiments the hollow-fiber membrane has a first zone of pores and a second zone of pores, wherein the first zone of pores adjoins the inner surface, and the second zone of pores adjoins the outer surface, and the density of pores in the second zone is greater than the density of pores in the first zone.

Average pore diameter or pore size of the pores can be determined, for example, by the method described in US 2017/0304780 (Asahi et al.) Average pore diameter or pore size of the pores can be determined by photographing a cross-section of the hollow fiber by a scanning electron microscope (SEM). For example, the photographing magnification is set at 50,000×, and the field of view is set on a cross-section perpendicular to the length direction of the hollow fiber or a cross-section parallel to the length direction and passing through the center of the hollow portion, horizontally to the cross-section. After photographing the set field of view, the photographing field of view is moved horizontally in the membrane thickness direction, and the next field of view is photographed.

This photographing operation is repeated until photographs of the cross-section of the membrane crossing from the outer surface to the inner surface are taken without a gap, and the obtained photographs are combined to obtain one membrane cross-section photograph. In this cross-section photograph, the average pore diameter of the pores in each area of (2 μm in the circumferential direction of the membrane)×(1 μm from the outer surface toward the inner surface side) from the outer surface toward the inner surface side is calculated, and the gradient structure of the membrane cross-section is quantified for each 1 μm from the outer surface toward the inner surface side. By such quantification, it can be determined as to whether or not the membrane has a gradient-type porous structure.

The average pore diameter or pore size can be calculated by a method using image analysis. The identification between a pore portion and a solid portion is based on their brightness, and a portion that cannot be identified and noise are corrected by a free hand tool. An edge portion forming the outline of a pore portion. After the binarization processing, the diameter of a pore is calculated from the area value of the pore assuming that the pore is a perfect circle. The calculation is carried out for all pores, and the average pore diameter is calculated for each area of 1 μm×2 μm. A pore portion that is located at the end of the field of view and is partially in the field of view is also counted (i.e. its diameter is calculated assuming that the area of a pore portion partially in the field of view is the area of one whole perfect circle).

Another embodiment of a hollow-fiber membrane according to the present disclosure is shown in FIG. 2. FIG. 2 illustrates a cross-section view of an exemplary hollow-fiber membrane 112. Hollow-fiber membrane 112 may have a continuous hollow lumen 116, which extends from one end to the other end of the fiber, an outer surface 118 facing outwards, which forms an outer side of the fiber; an inner surface 120 facing towards the hollow lumen 116, which defines the limits of the continuous hollow lumen 116; and an intermediate wall 122 having a wall thickness 126. Hollow-fiber membrane 112 may have a first cross section zone 128 that begins at the inner surface 120 and extends (in some embodiments, laterally) into the interior of the intermediate wall 122 terminating at an internal distance within the intermediate wall 122. In the first cross section zone 128, the pore size progressively decreases in the direction of the arrow (i.e. the pore size progressively decreases across the first cross section zone in the direction from the inner surface 120 to the outer surface 118 for a distance of intermediate between the inner and outer surface across the membrane wall (with the measurement of pore sizes being made along a vector that defines the shortest cross-section distance from the inner surface of the membrane to the outer surface of the membrane).

Hollow-fiber membrane 112 may have a second cross section zone 130 that begins where the first cross section zone terminates and extends (in some embodiments, laterally) to the outer surface 118 of the membrane. In the second cross section zone 130, the pore size progressively increases in the direction of the arrow (i.e. the pore size progressively increases across the second cross section zone in the direction from the termination of the first cross section zone in the interior of the wall to the outer surface). In some embodiments, the pore size at the outer surface 118 may be smaller than the pore size at the inner surface 112.

In some embodiments, in the first cross section zone 128, the pore size progressively decreases in the direction of the arrow (i.e. the pore size progressively decreases across the first cross section zone in the direction from the inner surface 120 to the outer surface 118 for a distance of about 10%, 15%, 20%, 25%, 30%, 35%, 40%, 45%, 50%, 55%, 60%, 65%, 70%, 75%, 80%, 85%, or 90% across the membrane wall (with the measurement of pore sizes being made along a vector that defines the shortest cross-section distance from the inner surface of the membrane to the outer surface of the membrane). In the second cross section zone 130, the pore size progressively increases in the direction of the arrow (i.e. the pore size progressively increases across the second cross section zone in the direction from the termination of the first cross section zone in the interior of the wall to the outer surface). The pore size at the outer surface 118 may be smaller than the pore size at the inner surface 112.

In some embodiments, the pore size progressively decreases in the direction of the arrow (i.e. the pore size progressively decreases across the first cross section zone in the direction from the inner surface 120 to the outer surface 118 for a distance of about 10%, 15%, 20%, 25%, 30%, 35%, 40%, 45%,50%, 55%, 60%, 65%, 70%, 75%, 80%, 85%, or 90% across the membrane wall (with the measurement of pore sizes being made along a vector that defines the shortest cross-section distance from the inner surface of the membrane to the outer surface of the membrane). In the second cross section zone 130, the pore size progressively increases in the direction of the arrow (i.e. the pore size progressively increases across the second cross section zone in the direction from the termination of the first cross section zone in the interior of the wall to the outer surface). The pore size at the outer surface 118 may be about 0.05 to 3 micrometers and the pore size at the inner surface 112 may be about 0.05 to 5 micrometers.

In some embodiments, the pore size progressively decreases in the direction of the arrow (i.e. the pore size progressively decreases across the first cross section zone in the direction from the inner surface 120 to the outer surface 118 for a distance of about 10%, 15%, 20%, 25%, 30%, 35%, 40%, 45%, 50%, 55%, 60%, 65%, 70%, 75%, 80%, 85%, or 90% across the membrane wall (with the measurement of pore sizes being made along a vector that defines the shortest cross-section distance from the inner surface of the membrane to the outer surface of the membrane). In the second cross section zone 130, the pore size progressively increases in the direction of the arrow (i.e. the pore size progressively increases across the second cross section zone in the direction from the termination of the first cross section zone in the interior of the wall to the outer surface). The pore size at the outer surface 118 may be about 0.05 to 3 micrometers and the pore size at the inner surface 112 may be about 0.05 to 5 micrometers, and the pore size at the pore size transition location may be about 0.015 to 0.035 micrometers.

The pore size transition location (i.e., the location where the first cross section zone ends and transitions into the start of the second cross section zone) may form at least a portion of a retention layer or retention zone in the hollow-fiber membrane. The retention layer or retention zone is the section of a hollow-fiber membrane with the greatest (i.e., maximum) capability or capacity to capture small contaminant components of the liquid sample when it is filtered through the membrane. Typically, the liquid sample to be filtered through the hollow-fiber membrane contains desired components that are preferably collected post-filtration in the filtrate and contaminant components that are preferably captured by the membrane. The retention layer or retention zone principally filters contaminants from the liquid sample based on differences in the size of contaminants and desired components. The desired component or components in the liquid sample are of a size that can pass through the retention layer or zone and be collected in the filtrate resulting in a purified liquid sample.

In some embodiments, the pore size progressively decreases in the direction of the arrow (i.e. the pore size progressively decreases across the first cross section zone in the direction from the inner surface 120 to the outer surface 118 to a distance 3 to 50 micrometers from the outer surface. (with the measurement of pore sizes being made along a vector that defines the shortest cross-section distance from the inner surface of the membrane to the outer surface of the membrane). In the second cross section zone 130, the pore size progressively increases in the direction of the arrow (i.e. the pore size progressively increases across the second cross section zone in the direction from the termination of the first cross section zone in the interior of the wall to the outer surface). The pore size at the outer surface 118 may be smaller than the pore size at the inner surface 112.

In some embodiments, the hollow-fiber membrane wall thickness is 30 to 100, 40 to 90, or 50 to 65 micrometers and the pore size of the membrane at the inner surface facing the lumen progressively decreases across the first cross section zone in the direction from the inner surface to the outer surface to a distance 3 to 50 micrometers from the outer surface. (with the measurement of pore sizes being made along a vector that defines the shortest cross-section distance from the inner surface of the membrane to the outer surface of the membrane). In the second cross section zone 130, the pore size progressively increases in the direction of the arrow (i.e. the pore size progressively increases across the second cross section zone in the direction from the termination of the first cross section zone in the interior of the wall to the outer surface). The pore size at the outer surface 118 may be smaller than the pore size at the inner surface 112.

In some embodiments, the pore size at the outer surface is 0.05 to 3 micrometers, the pore size at the inner surface is about 0.05 to 5 micrometers, and the pore size at the pore size transition location being about 0.015 to 0.035 micrometers.

In some embodiments, hollow-fiber membrane wall thickness is 30 to 100 micrometers, the pore size at the outer surface is 0.05 to 3 micrometers, the pore size at the inner surface is about 0.05 to 5 micrometers, and the minimum or smallest pore size in the membrane is 0.015 to 0.035 micrometers.

The aromatic sulfone polymer of the present disclosure, e.g. polysulfones, polyethersulfones, polyphenylene sulfones, polyarylethersulfones or copolymers or modifications of these polymers or mixtures of these polymers can be used. In a preferred embodiment, the aromatic sulfone polymer can be a polysulfone or a polyethersulfone with the repeating molecular units shown in formulas (I) and (II) as follows:

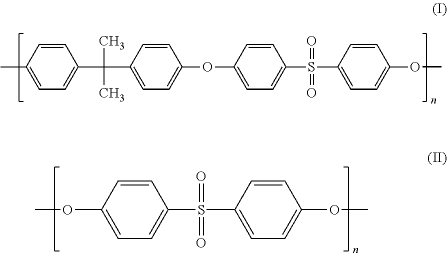

More preferably, a polyethersulfone according to formula (II) is used as the aromatic sulfone polymer, because this has lower hydrophobicity than, for example, the polysulfone. The polysulfone may have a molecular weight of about 72 kg/mol.

In some embodiments, the polyoxazoline of the present disclosure can be a poly(2-oxazoline). Poly(2-oxazolines) can be prepared by cationic ring opening polymerization reactions of various 2-oxazoline monomers. Polymerization of 2-alkyl substituted 2-oxazoline monomers provides poly(2-alkyl-2-oxazolines)

In some embodiments, the poly(2-oxazoline) of the present disclosure can be poly(2-ethyl-2-oxazoline) (PEtOx). Poly(2-oxazolines) have high potential for protein repulsion. The residual groups of poly(2-oxazolines) can be changed, to alter the properties of the polymers, e.g. from hydrophilic to hydrophobic. The poly(2-oxazolines) may have a molecular weight of from about 25 kg/mol to about 500 kg/mol. The poly(2-oxazoline) may have a molecular weight of from about 50 kg/mol.

The poly(2-ethyl-2-oxazoline) can have a molecular weight of from about 25 kg/mol to about 500 kg/mol. The poly(2-ethyl-2-oxazoline) can have a molecular weight of from about 25 kg/mol to about 100 kg/mol. The poly(2-ethyl-2-oxazoline) can have a molecular weight of about 50 kg/mol.

The poly(2-oxazoline) can be present in a concentration of 0.5 to 30 wt. %, 1 to 30 wt. %, 5 to 30 wt. %, or 10 to 30 wt. % relative to the weight of the membrane. The poly(2-oxazolines) can be present in a concentration of more than 0.5 wt. %, more than 1 wt. %, more than 2 wt. %, more than 3 wt. %, more than 4 wt. %, more than 5 wt. %, more than 6 wt. %, more than 7 wt. %, more than 8 wt. %, more than 9 wt. %, more than 10 wt. %, more than 15 wt. %, or more than 20 wt. % relative to the weight of the membrane. The poly(2-oxazolines) can be present in a concentration of less than 30 wt. %, less than 28 wt. %, less than 25 wt. %, less than 23 wt. %, less than 20 wt. %, less than 15 wt. %, or less than 10 wt. % relative to the weight of the membrane.

The aromatic sulfone polymer and the poly(2-oxazoline) may be distributed throughout the membrane. The aromatic sulfone polymer and the poly(2-oxazoline) may be evenly distributed throughout the membrane. The aromatic sulfone polymer and the poly(2-oxazoline) may be uniformly distributed throughout the membrane.

In some embodiments, the polyoxazoline may be distributed throughout the membrane. The poly(2-oxazoline) may be distributed throughout the membrane. The poly(2-oxazoline) may be evenly distributed throughout the membrane. In some embodiments, the poly(2-oxazoline) may be uniformly distributed throughout the membrane. Poly(2-ethyl-2-oxazoline) may be distributed throughout the membrane. In some embodiments, poly(2-ethyl-2-oxazoline may be evenly distributed throughout the membrane. In some embodiments, poly(2-ethyl-2-oxazoline may be uniformly distributed throughout the membrane. In some embodiments, poly(2-oxazoline) may not be evenly distributed throughout the membrane. In some embodiments, poly(2-ethyl-2-oxazoline) may not be uniformly distributed throughout the membrane. For example, the concentration of poly(2-ethyl-2-oxazoline) adjourn the outer surface may be more than the concentration of poly(2-ethyl-2-oxazoline) adjourn the inner surface.

In some embodiments, the polymeric blend may further include an additional hydrophilic polymer. Exemplary hydrophilic polymer can include polyvinylpyrrolidone, polyethylene glycol, glycerol, polyvinyl alcohol, polyglycol monoester, polysorbitate, carboxymethylcellulose, polyacrylic acid, polyacrylate, or a modification or a copolymer of these polymers. In some embodiments, the hydrophilic polymer can be polyethylene glycol. In some embodiments, the polymeric blend does not comprise polyvinylpyrrolidone. In some embodiments, the polymeric blend can be a hydrophobic polymeric blend.

In some embodiments, the hydrophilic polymer can be present in a concentration of 1 to 75 wt. % relative to the weight of the membrane. In some embodiments, the polymeric blend can include more than 7 wt. %, more than 10 wt. %, more than 20 wt. %, more than 30 wt. %, more than 40 wt. %, more than 50 wt. %, more than 60 wt. %, more than 70 wt. %, more than 80 wt. %, or more than 90 wt. % of polyvinylpyrrolidone. In some embodiments, the polymeric blend can include less than 3 wt. %, less than 2 wt. % or less than 1 wt. % of polyvinylpyrrolidone.

In some embodiments, the polymeric blend may include a solvent and non-solvents. Exemplary blends can include glycol, glycerol, butyrolactone, ε-caprolactam, N-methyl pyrrolidone, water or combination thereof.

It is also preferred that the wall thickness of the hollow-fiber membranes as disclosed herein is in the range of from 10 to 400 μm, from 20 to 300 μm, from 30 to 200 μm, or from 40 to 80 μm. At a wall thickness less than 20 μm, the mechanical properties of the hollow-fiber membrane may fall below a certain desirable level, while at wall thicknesses above 400 μm, the trans membrane flow decreases. Similarly, in order to achieve a desirable flow through the lumen of the hollow-fiber membranes according to the present disclosure, particularly, a favorable pressure drop, it is preferred that the inside diameter of the hollow-fiber membranes as described herein is in the range of from 50 to 800 μm, from 50 to 700 μm, from 50 to 600 μm, from 100 to 500 μm, from 100 to 400 μm, or from 100 to 300 μm.

The hollow-fiber membranes according to the present invention preferably exhibit a trans membrane flow for water of at least 0.01 mL/(cm²·min·bar), preferably at least 0.1 mL/(cm²·min·bar), more preferably at least 0.15 mL/(cm²·min·bar), and even more preferably at least 0.2 mL/(cm²·min·bar). This ensures an adequate and stable filtration capacity in the application. It is further preferred that the hollow-fiber membranes as disclosed herein exhibit a trans membrane flow for water in the range of from 0.01 to 10 mL/(cm²·min·bar), preferably from 0.15 to 5 mL/(cm²·min·bar), and more preferably from 0.1 to 3 mL/(cm²·min·bar). Trans membrane flows in these ranges allow for adequate and stable filtration capacity in suitable applications without deteriorating the retention capacity or compromising the mechanical stability. The trans membrane flow is preferably determined as described in the experimental section.

The hollow-fiber membranes according to the present disclosure can be made by methods disclosed in WO 2019/229667 A1 (Malek et al.), which is incorporated herein by reference in its entirety into this disclosure. In some embodiments, the hollow-fiber membranes can be made from a homogeneous spinning solution of an aromatic sulfone polymer and a poly(2-oxazoline), and a bore liquid. The bore liquid can include water, a solvent and a non-solvent. Accordingly, the present disclosure further provides a method for producing a hollow-fiber membrane, comprising the following steps: providing a spinning solution comprising an aromatic sulfone polymer and a polyoxazoline, and a bore liquid comprising water, a solvent and a non-solvent; and spinning an aromatic sulfone polymer and poly(2-oxazoline) hollow-fiber with a spinneret outer diameter in the range of from 300 to 1000 μm, a spinneret needle outer diameter in the range of from 200 to 1000 μm and a spinneret needle inner diameter in the range of from 100 to 1000 μm.

In some embodiments, the spinning solution can further include a hydrophilic polymer. Long-chain polymers are advantageously employed as at least one hydrophilic polymer that exhibit a compatibility with the hydrophobic aromatic sulfone polymer. The aromatic sulfone polymers have repeating polymer units that in themselves are hydrophilic. The hydrophilic polymer is preferably polyvinylpyrrolidone, polyethylene glycol, polyvinyl alcohol, polyglycol monoester, a polysorbitate such as polyoxyethylene sorbitan monooleate, carboxymethylcellulose or a modification or copolymer of these polymers. Polyvinylpyrrolidone and polyethylene glycol are particularly preferred.

In some embodiments, the spinning solutions includes a polyethylene glycol PEG). In some embodiments, the polyethylene glycol in the spinning solution can have a molecular weight (MW) from about 100 to about 1,800 g/mol. In some embodiments, the polyethylene glycol in the spinning solution can have a molecular weight (MW) of about 200, 400, 500, 600, 1000, 1200, or 1,500 g/mol.

Within the context of the present disclosure, at least one hydrophilic polymer can also comprise mixtures of different hydrophilic polymers. The hydrophilic polymer can, for example, be a mixture of chemically different hydrophilic polymers or of hydrophilic polymers with different molecular weights, e.g. a mixture of polymers whose molecular weight differs by a factor of 5 or more. Preferably, at least one hydrophilic polymer comprises a mixture of polyvinylpyrrolidone or polyethylene glycol with a hydrophilically modified aromatic sulfone polymer. It is also preferred that the hydrophilically modified aromatic sulfone polymer is a sulfonated aromatic sulfone polymer, in particular a sulfonated modification of the hydrophobic aromatic sulfone polymer employed in the membrane and in the method according to the present disclosure. Mixtures of polyether sulfone, sulfonated polyether sulfone and polyvinylpyrrolidone can be particularly advantageously employed. As a result of the presence of a hydrophilically modified aromatic sulfone polymer, hollow-fiber membranes with particularly stable hydrophilic properties in the application are obtained. The hydrophilic polymer can be present from 5 to 50 wt. %, relative to the weight of the solution.

After preferably degassing and filtration to remove gasses and undissolved particles, the homogeneous spinning solution is extruded through the annular gap of a conventional hollow-fiber die in conjunction with a bore fluid to produce a hollow fiber. A bore liquid, i.e. an interior filler that is a coagulation medium for the aromatic sulfone polymer and at the same time stabilizes the lumen of the hollow-fiber, is extruded through the central nozzle opening arranged coaxially to the annular gap in the hollow-fiber die. Within the present disclosure, the terms “hollow-fiber die” and “spinneret” may be used interchangeably. The bore liquid may comprise water and glycerol but may also comprise additional ingredients and/or solvents, for example, polyethylene glycol (PEG). Preferably, the bore liquid further comprises non-solvents for the membrane-forming polymer such as water, low-molecular polyethylene glycols with a mean molecular weight of less than 1000 Daltons or low-molecular alcohols such as ethanol or isopropanol, and/or protic solvents such as ε-caprolactam. Preferably, the bore liquid comprises water, N-methylpyrrolidone and polyethylene glycol. Solvent can be present from 5 to 70 wt. %, relative to the weight of the solution.

The solvent system to be employed must be matched to the aromatic sulfone polymer employed and to the poly(2-oxazoline) so that a homogeneous spinning solution can be produced. The solvent system preferably comprises polar, aprotic solvents such as dimethylformamide, dimethylacetamide, dimethyl sulfoxide, N-methyl pyrrolidone or their mixtures, or protic solvents such as ε-caprolactam. Furthermore, the solvent system can contain up to 70 wt. % latent solvent, whereby in the context of the present invention a latent solvent is understood as a solvent that poorly dissolves the sulfone polymer or dissolves it only at elevated temperature. In cases where ε-caprolactam is used as a solvent, butyrolactone, propylene carbonate or polyalkylene glycol can be employed, for example. In addition, the solvent system can contain non-solvents for the membrane-forming polymer such as water, glycerin, low-molecular polyethylene glycols with a mean molecular weight of less than 1000 Daltons or low-molecular alcohols such as ethanol or isopropanol. Preferably, the solvent system contains N-methyl pyrrolidone.

In one embodiment, the spinning solution includes an aromatic sulfone polymer, poly(2-oxazoline), a polyethylene glycol, N-methylpyrrolidone, and water. In another embodiment, the spinning solution includes a polyethersulfone, poly(2-ethyl-2-oxazoline), polyethylene glycol, N-methylpyrrolidone, and water. In still another embodiment, the spinning solution includes a polyethersulfone, poly(2-ethyl-2-oxazoline), PEG200 or PEG1500, N-methylpyrrolidone, and water.

The width of the annular gap and the inside diameter of the central nozzle opening were selected according to the desired properties of the hollow-fiber membrane according to the present disclosure. That is, the spinneret exhibits a spinneret outer diameter for dope in the range of from 300 to 1000 μm, a spinneret needle outer diameter in the range of from 200 to 1000 μm and a spinneret needle inner diameter in the range of from 100 to 1000 μm.

After leaving the hollow-fiber die (i.e. the spinneret) and before entering a coagulation medium, it is preferred that the hollow-fiber passes through a climate-controlled zone with defined climatic conditions. The climate-controlled zone can thereby take the form of e.g. an encapsulated chamber. For technical reasons it may be necessary for an air gap to exist between the hollow-fiber die and the climate-controlled zone. This gap should, however, advantageously be as small as possible; the climate-controlled zone preferably directly follows the hollow-fiber die.

In this regard, it is preferred that the hollow-fiber has a retention time in the climate-controlled zone of 0.5 to 10 s, whereby the climate-controlled zone contains air with a relative humidity of 20 to 95% and a temperature of 25 to 75° C. It is preferred that the retention time of the hollow-fiber in the climate-controlled zone is 0.5 to 5 s. In order to establish stable conditions in the climate-controlled zone, the air preferably flows through the climate-controlled zone with a velocity of less than 0.5 m/s and particularly preferably with a velocity in the range from 0.15 to 0.35 m/s.

In one embodiment, the climate-controlled zone contains air with a relative humidity of 20 to 95% and a temperature of 25 to 75° C. In one embodiment, the climate-controlled zone contains air with a relative humidity of 60 to 75% and a temperature of 30 to 50° C. In one embodiment, the climate-controlled zone contains air with a relative humidity of 75 to 90% and a temperature of 30 to 50° C. In one embodiment, the climate-controlled zone contains air with a relative humidity of 60 to 75% and a temperature of 50 to 70° C. In one embodiment, the climate-controlled zone contains air with a relative humidity of 75 to 90% and a temperature of 50 to 70° C.

As the hollow-fiber is directed through the climate-controlled zone set to the climatic conditions preferred in the method according to the present disclosure, a precoagulation of the hollow-fiber is induced by absorption on the outside of the hollow-fiber of the water vapor acting as the non-solvent, before the coagulation of the hollow fiber. Simultaneously, the retention time should be set within the range preferred in the method according to the present disclosure. These measures influence the formation of the outer layer of the hollow-fiber membrane according to the invention so that in some embodiments, the outer layer can obtain an essentially isotropic structure.

After passing through the climate-controlled zone, the precoagulated hollow-fiber is directed through an aqueous coagulation medium preferably conditioned to 20 to 90° C. in order to complete the formation of the membrane structure. The coagulation medium is preferably conditioned to a temperature in the range from 20 to 90° C. Preferably, the coagulation medium, such as precipitation bath, is water or a water bath.

In the coagulation medium, the membrane structure is first precipitated to such an extent that it already has sufficient stability and can be diverted over e.g. deflection rollers or similar means in the coagulation medium. During the further course of the process, the coagulation is completed and the membrane structure stabilized. An extraction of the solvent system and soluble substances takes place here at the same time. In general, a large proportion of the hydrophilic polymer, is extracted from the membrane structure, so that the coagulation baths serve at the same time as washing or extraction baths. Water is preferably employed as a coagulation or washing medium in the coagulation or washing baths.

After extraction, the hollow-fiber membrane can be dried. The dried membrane can be then coiled. The hollow-fiber membrane according to the present disclosure may then be texturized (if necessary) to improve the exchange properties of the hollow-fiber membrane in the bundle. Finally, the hollow-fiber membrane can be processed using conventional methods, e.g. wound onto a coil or formed directly into bundles with a suitable fiber count and length. Before production of the bundles, supplementary threads, e.g. in the form of multifilament yarns, can be added to the hollow-fiber membranes in order to ensure a spacing of the hollow-fiber membranes relative to one another and a better flow around the individual hollow-fiber membranes in the bundle.

According to the present disclosure, the concentration of the sulfone polymer in the spinning solution is preferably in the range of from 10 to 35 wt. %. Below a concentration of 10 wt. %, disadvantages may arise in particular with respect to the mechanical stability of the hollow-fiber membranes obtained. The sulfone polymer can also contain additives such as antioxidants, nucleating agents, UV absorbers, etc. to selectively modify the properties of the membranes. The concentration of poly(2-oxazolines) in the spinning solution can be in the range of from 5 to 30 wt. %.

In some embodiments, the spinning solution can have 10-35 wt. %, relative to the weight of the solution, of the aromatic sulfone polymer; 5-30 wt. %, relative to the weight of the solution, of a poly(2-oxazoline); 25-70 wt. %, relative to the weight of the solution, of a solvent; and 5-45 wt. %, relative to the weight of the solution, of a hydrophilic polymer; 0-10 wt. % of a sulfonated aromatic sulfone polymer. In some embodiments, the spinning solution can have 20-30 wt. %, relative to the weight of the solution, of the aromatic sulfone polymer; 7-15 wt. %, relative to the weight of the solution, of a poly(2-oxazoline); 30-40 wt. %, relative to the weight of the solution, of a solvent; and 25-50 wt. %, relative to the weight of the solution, of a hydrophilic polymer.

In some embodiments, the spinning solution can have 10-35 wt. %, relative to the weight of the solution, of a polyethersulfone; 10-30 wt. %, relative to the weight of the solution, of poly(2-ethyl-2-oxazoline); 50-70 wt. %, relative to the weight of the solution, of N-methylpyrrolidone.

In some embodiments, the spinning solution can have 10-35 wt. %, relative to the weight of the solution, of a polyethersulfone; 5-20 wt. %, relative to the weight of the solution, of poly(2-ethyl-2-oxazoline); 20-70 wt. %, relative to the weight of the solution, of N-methylpyrrolidone; and 5-40 wt. %, relative to the weight of the solution, of a polyethylene glycol.

In some embodiments, the spinning solution can have 5-18 wt. %, relative to the weight of the solution, of poly(2-ethyl-2-oxazoline).

In some embodiments, the spinning solution can have 25-75 wt. %, relative to the weight of the solution, of N-methylpyrrolidone. In some embodiments, the spinning solution can have 25-50 wt. %, relative to the weight of the solution, of N-methylpyrrolidone. In some embodiments, the spinning solution can have 50-75 wt. %, relative to the weight of the solution, of N-methylpyrrolidone.

In some embodiments, the polyethylene glycol component of the spinning solution can have a molecular weight of 200 g/mol (PEG200), a molecular weight of 400 g/mol (PEG400), a molecular weight of 600 g/mol (PEG600), a molecular weight of 1000 g/mol (PEG1000), a molecular weight of 1200 g/mol (PEG1200), or a molecular weight of 1500 g/mol (PEG1500).

In some embodiments, the polyethylene glycol component of the spinning solution can have a molecular weight of 200-600 g/mol. In some embodiments, the polyethylene glycol component of the spinning solution can have a molecular weight of 500-1000 g/mol. In some embodiments, the polyethylene glycol component of the spinning solution can have a molecular weight of 1000-1500 g/mol.

The invention provides polymeric membranes with superior protein repelling characteristics. Therefore, these membranes block more slowly, show a higher throughput behavior and thus a longer lifetime. These membranes further exhibit an asymmetrical structure that is promising for the preparation of highly selective membranes. The protein repelling characteristics of the membranes can provide better filtration characteristics due to less fouling and higher throughput.

In some embodiments, the hollow-fiber membrane of the present discourse can be used for multiple extracorporeal blood purification procedures including dialysis. In some embodiments, the hollow-fiber membrane of the present discourse can be suitable for use in applications in the field of filtration. Due to the unique combination of properties of the hollow-fiber membrane as described herein, preferably obtained from the method as described herein, the present disclosure further provides a use of the membranes as described herein for filtration of liquids, for example, microfiltration or ultrafiltration. “Microfiltration” and “ultrafiltration” have the meaning common in the art. Preferably, the use as described herein comprises clarification and/or purification of liquid media, in particular aqueous liquids. In some embodiments, the liquids that can be filtered by the hollow-fiber membrane of the present discourse, can include a biological product selected from adeno-associated virus (AAV) capsids, viruses, virus like particles. The hollow-fiber membrane can have a more than 80%, 85%, 90%, 95%, or 96% yield of adeno-associated virus (AAV) capsids, while removing more than 4 log reduction value (LRV) of contaminating bacteriophages or viruses that are 35-40 nm or greater and 5 log, 6 log or 7 log reduction value (LRV) of contaminating bacteriophages or viruses, for example, mammalian viruses, that are 50 nm or greater. It can provide a high transmission (yield) of AAV and can be operated at a variety of transmembrane pressures, for example from 7 to 30 psi. The hollow-fiber membrane can be operated using either constant flow rate or a constant pressure. These membrane attributes enable faster processing times and more flexibility in the processing equipment that is used to perform the filtration, and the ability to be operated under a variety of conditions.

The following working examples are intended to be illustrative of the present disclosure and not limiting.

### EXAMPLES

Objects and advantages of this invention are further illustrated by the following examples, but the particular materials and amounts thereof recited in these examples, as well as other conditions and details, should not be construed to unduly limit this invention.

The following abbreviations are used herein: mL=milliliters, L=liters, kg=kilograms, g=grams, mg=milligrams, m=meters, cm=centimeters, mm=millimeters, nm=nanometers, s=seconds, min=minutes, hr=hours, psi=pounds per square inch, and wt. %=percent by weight.

Scanning electron microscope (SEM) images were obtained using an FEI 250 scanning electron microscope with xT Microscope Control operating software (Thermo Fisher Scientific, Waltham, MA), or a Coxem EM-30AX scanning electron microscope with NanoStation operating software (Coxem Company, Daejeon, Korea).

TABLE 1
Materials List

| Description (Abbreviation) | Source |
|---|---|
| Polyethersulfone (PES), (ULTRASON | BASF SE, Ludwigshafen, |
| E6020), (MW = 72,000 g/mol) | Germany |
| Poly(2-ethyl-2-oxazoline) (PEtOx) | Kremer Pigmente GmbH & Co, |
| (MW = 50,000 g/mol) | KG, Aichstetten, Germany |
| Poly(ethylene glycol) 200 (PEG200) | VMP Chemiekontor GmbH, |
| (MW = 200 g/mol) | Siegburg, Germany |
| Poly(ethylene glycol) 1500 (PEG1500) | Clariant GmbH, Burgkirchen, |
| (MW = 1,500 g/mol) | Germany |
| N-Methylpyrrolidone (NMP) | Ashland Inc., Wilmington, DE |

### Method A. Method for Determining Transmembrane Flow (TMF)

A hollow-fiber membrane test module was prepared by placing ten hollow-fiber membranes (10 cm in length) in a straight, cylindrical polycarbonate tube (inner diameter of 8 mm and a length of 60 mm). The tube had a side-positioned outlet located about midway between the two ends of the cylinder. The hollow-fiber membranes were imbedded in the tube using hot melt glue at both ends of the tube. After solidification, the protruding ends of the hollow-fiber membranes and excess glue were removed using a razor blade. The openings of the membranes were visually inspected and only modules in which all of the hollow-fiber membranes had open and unobstructed lumen portions were used. The polycarbonate tube was capped at each end with caps having an open port for attachment to flexible tubing. The finished test module was attached to a stand, placed in a vertical orientation, filled with water, and connected to a measuring system.

The measuring system included a pressure pot filled with water that was connected to one end of the test module with flexible tubing; two pressure gauges (the first pressure gauge located between the pressure pot and the test module and the second pressure gauge positioned downstream from the opposite end of the module); and a flush valve positioned downstream from the module and the second pressure gauge. The measuring system also included a heater that the water passed through that was located between the pressure pot and the first pressure gauge. The heater warmed the water to 25° C.

A pressure of 5.8 psi was applied to the pressure pot. At the start of the test, the air in the system was displaced by closing the side outlet and opening the flush valve. The flush valve was then closed and the side outlet opened to operate the module in a dead-end filtration configuration. The water flowed from the pressure pot through the lumen of the membranes, filtering through the membrane walls, and exiting the module through the side outlet into a first collection vessel. The module was flushed for four minutes with water and collected through the side outlet into a first collection vessel. After the four minute flush, the first collection vessel was replaced with a tared second collection vessel. The filtered water was collected in the second collection vessel for 60 seconds. The amount of water collected in the second vessel was determined using a digital balance. The differential pressure was determined by reading the difference between the two pressure gauges.

Based on the dimensions of the membrane, the differential pressure, and the weight of the water, the transmembrane flow (TMF) was calculated according to Equation 1.

$$TMF [ \frac{mL}{{cm}^{2} \cdot min \cdot bar} ] = \frac{{m}_{W} / {ρ}_{w}}{[ Δ t \cdot {A}_{M} \cdot Δ p ]} . \qquad Equation 1$$

where:
m_{W}=amount of water (in grams) passed through the membrane sample during the measuring period
ρ_{w}=Density of water at 25° C.
Δt=Measuring time (minutes)
A_{M}=Total inner surface area of hollow membrane in the test module
Δp=Differential pressure along the length of the test module (bar)

### Method B. Method for Determining the Viscosity of a Spinning Solution (Polymeric Blend)

The viscosity of a casting solution was determined at 60° C. and a shear rate of 10 s⁻¹ using a HAAKE RheoStress 1 rheometer (Thermo Fisher Scientific, Waltham, MA) with a Z20DIN sensor device (Thermo Fisher Scientific).

### Method C. Method for Determining Protein Throughput Using Bovine Serum Albumin (BSA)

A 5 mg/mL solution of bovine serum albumin (BSA) (Millipore Sigma, Burlington, MA) was prepared by vigorously mixing the protein with phosphate buffer (pH 7.4, 4 mS/cm). The solution was filtered through a 0.2 micron filter and used within 8 hours of preparation. The concentration of the BSA solution was verified using ultraviolet spectroscopy at 280 nm.

Hollow-fiber membrane test modules were prepared and tested according to the following procedure. Polycarbonate tubes with lengths of 50 mm and inner diameters of 4 mm were used. A single hole was drilled in the side of each tube about 15 mm from one end. An open-bore connector was attached to the hole using a uv/visible light cured adhesive to form a side port. About 25-30 hollow-fibers (15 cm length) were placed in each tube. The inserted hollow-fibers were cut with a razor blade to provide an overhang of hollow-fibers of about 15 mm at each end of the tube. The overhanging hollow-fibers were sealed with wax and then potted in the tube using a polyurethane resin. After curing for 24 hours, the protruding ends were removed using a razor blade. The openings of the membranes were inspected using a microscope and only tubes in which all of the hollow-fiber membranes had open and unobstructed lumen portions were used. The total inner hollow-fiber surface area (i.e. total lumen surface area) was about 5-6 cm² for the 50 mm tubes.

The membrane test module was mounted vertically, parallel to a vertically mounted pressure pot. A three-way valve was located at the bottom of the pressure pot. MASTERFLEX tubing (size 14, Cole-Parmer, Vernon Hills, IL) was used to connect the three-way valve to the lower end of the vertically mounted membrane test module. The pressure pot was initially filled with ultrapure water (obtained from a MILLI-Q water system, MilliporeSigma), sealed, and pressurized to approximately 5 psi. The three-way valve between the pressure pot and test module was opened allowing the water to flow into the lumens of the hollow-fibers and out the open upper end of the tube. When the lumens of the hollow-fibers were filled with water, the upper end of the test module was capped. The pressure was gradually increased to 30 psi. The side port on the test module was used to allow the filtrate to exit the module into a first collection vessel. The water was filtered through the hollow-fiber modules for a minimum of 10 minutes at 30 psi. After the initial water flushing period, the three-way valve at the bottom of the pressure pot was closed and any remaining water in the pressure pot was removed.

The pressure pot was depressurized and filled with the 5 mg/mL BSA solution. The pressure pot was then sealed, pressurized to 30 psi, and the three-way valve was opened. The filtrate was collected in a tared, second collection vessel that was placed on a digital balance. The BSA solution was filtered through the membrane for a minimum of 20 minutes. The amount of BSA solution that passed through the membrane during the filtration time was recorded as the mass of BSA solution filtered (kg) divided by the filtration area of the hollow fibers (surface area of inside fiber walls) in the specified filtration time (kg/(m²·hr)).

### Method D. Phi-X174 Phage Culture Preparation

Phi-X174 bacteriophage (ATCC 13706-B1) was obtained from ATCC (Manassas, VA). The phage culture was produced by growing a 1 L culture of E. coli (ATCC 13706) in CRITERION Nutrient Broth (Hardy Diagnostics, Santa Maria, CA) plus 5% sodium chloride at 37° C. with mixing at 210 revolutions per minute (rpm) to an OD of 0.45. The culture was inoculated with about 1,000 plaque-forming units (pfu) of Phi-X174 phage. The inoculated culture was grown for an additional 4 hours at 37° C. with mixing at 210 rpm. The inoculated Phi-X174 culture was then purified using anion exchange chromatography. The purified Phi-X174 was sterile filtered through a 0.2 micron syringe filter. The phage concentration was determined per Method F and was stored at 4° C.

### Method E. Filtration of Phi-X174 Phage Solutions

Hollow-fiber membrane test modules were prepared and tested according to the following procedure. Polycarbonate tubes with lengths of 90 mm and inner diameters of 4 mm were used. A single hole was drilled in the side of each tube about 15 mm from one end. An open-bore connector was attached to the hole using a uv/visible light cured adhesive to form a side port. About 25-30 hollow-fibers (15 cm length) were placed in each tube. The inserted hollow-fibers were cut with a razor blade to provide an overhang of hollow-fibers of about 15 mm at each end of the tube. The overhanging hollow-fibers were sealed with wax and then potted in the tube using a polyurethane resin. After curing for 24 hours, the protruding ends were removed using a razor blade. The openings of the membranes were inspected using a microscope and only tubes in which all of the hollow-fiber membranes had open and unobstructed lumen portions were used. The total inner hollow-fiber surface area (i.e. total lumen surface area) was about 13 cm² for the 90 mm tubes.

The membrane test module was mounted vertically, parallel to a vertically mounted pressure pot. A three-way valve was located at the bottom of the pressure pot. MASTERFLEX tubing (size 14, Cole-Parmer) was used to connect the three-way valve to the lower end of the vertically mounted membrane test module. The pressure pot was initially filled with ultrapure water (obtained from a MILLI-Q water purification system, EMD Millipore, Burlington, MA), sealed, and pressurized to approximately 5 psi. The three-way valve at the bottom of the pressure pot was opened allowing the water to flow into the lumens of the hollow-fibers and out the upper end of the tube. When the lumens of the hollow-fibers were filled with water, the upper end of the test module was capped and the pressure was gradually increased to 30 psi. The side port on the test module was used to allow the filtrate to exit the module. The filtrate was collected in a beaker placed on a scale. Ultrapure water was filtered through the test modules for a minimum of 10 minutes at 30 psi. After the initial water flush the three-way valve at the bottom of the pressure pot was closed and any remaining water in the pressure pot was removed.

Phi-X174 phage was spiked into phosphate buffer (pH 7.4, 4 mS/cm) at a concentration of 10⁷ pfu/mL and 150 mL of the phage solution was added to the pressure pot. The pressure pot was sealed, pressurized to 30 psi, and the three-way valve at the bottom of the pressure pot was opened. The filtrate was collected in a sterile container placed on a scale. At the end of the filtration, the filtrate container was capped and stored at 4° C. until the phage concentration could be determined.

### Method F. Determination of Phi-X174 Phage Concentration

The phage concentration of filtrate samples, feed solutions, and Phi-X174 culture preparations was determined using the following procedure. The solutions of interest were serially diluted (10-fold). Top agar (CRITERION Nutrient Broth (Hardy Diagnostics) with 0.9% agar, 2.5 mL) was mixed with 50 microliters of E. coli (ATCC 13706) culture (in CRITERION Nutrient Broth plus 5% sodium chloride grown at 37° C. with shaking at 210 rpm overnight) and 100 microliters of diluted Phi-X174 phage. The mixture was poured on top of a standard nutrient agar plate (CRITERION nutrient broth with 1.5% agar) and incubated for 3-4 hours at 37° C. Following incubation, the plaque-forming units (pfu) were counted. The number of pfu was correlated with phage particle number. The phage particle concentration (particles/mL) was calculated from the pfu count adjusted for dilution. Log reduction values (LRV) were determined by the difference in the number of plaques present in the feed solution and the number of plaques present in the filtrate (see Equation 1).

### Method G. T7 Phage Culture Preparation

T7 bacteriophage (ATCC BAA-1025-B2) was obtained from ATCC (Manassas, VA). The phage culture was produced by growing a 1 L culture of E. coli BL21 (ATCC BAA-1025) in CRITERION tryptic soy broth (Hardy Diagnostics, Santa Maria, CA) plus 5% sodium chloride at 37° C. with mixing at 210 rpm to an OD (optical density) of 0.45. The culture was inoculated with about 1,000 pfu of T7 phage. The inoculated culture was grown for an additional 4 hours at 37° C. with mixing at 210 rpm. The inoculated T7 culture was then filtered through a 0.2 micron PES filter and was stored at 4° C. The phage concentration was determined per Method H.

### Method H. Determination of T7 Phage Concentration

The phage concentration of filtrate samples, feed solutions, and T7 culture preparations was determined using the following procedure. The solutions of interest were serially diluted (10-fold). Top agar (CRITERION tryptic soy broth (Hardy Diagnostics) with 0.9% agar, 2.5 mL) was mixed with 50 microliters of E. coli BL21 (ATCC BAA-1025) culture (in CRITERION tryptic soy broth grown at 37° C. with shaking at 210 rpm overnight) and 100 microliters of diluted T7 phage solution. The mixture was poured on top of a standard tryptic soy agar plate (CRITERION tryptic soy broth with 1.5% agar) and incubated for 3-4 hours at 37° C. Following incubation, the pfu were counted. The number of pfu was correlated with phage particle number. The phage particle concentration (particles/mL) was calculated from the pfu count adjusted for dilution. Log reduction values (LRV) were determined by the difference in the number of plaques present in the feed solution and the number of plaques present in the filtrate (see Equation 1). A designation of “>” for a reported LRV indicates that no pfu were observed for any of the serial dilution samples of the filtrate.

$$LRV = log 10 [ \frac{concentration of Phage in the Feed Solution}{concentration of Phage in the Filtrate} ] \qquad Equation 1$$

### Example 1

A spinning solution was prepared by vigorously mixing 19 wt. % polyethersulfone, 13 wt. % poly(2-ethyl-2-oxazoline), 63 wt. % N-methylpyrrolidone, and 5 wt. % water at a temperature of about 55° C. The resulting spinning solution was cooled to about 50° C., filtered, and then degassed. A temperature-controlled spinneret (35° C.) having an outer diameter for dope of 0.41 mm, a needle outer diameter of 0.3 mm, and a spinneret needle inner diameter of 0.15 mm was used. The spinneret was fixed at a distance of 60 cm above the precipitation bath.

Using the above-mentioned spinning solution and a mixture of NMP:water (53:47) as the bore liquid in the spinneret needle of the spinneret, a hollow fiber was generated. The hollow fiber was transferred through a climate-controlled zone conditioned to a temperature of 25-75° C. and 20-95% relative humidity. Next, the hollow fiber was transferred into the water-containing precipitation bath heated to about 71° C., thereby fixing the membrane structure. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at about 90° C. for one hour. The resulting hollow-fiber membranes had a physical inner diameter of about 250 micrometers and a wall thickness of about 70 micrometers, a zone with minimum pore size located adjoining the inner surface, and a zone with maximum pore size located approximately in the middle of the membrane. In addition, pores on the outer surface were larger in size than the pores on the inner surface. The transmembrane flow (TMF) was measured to be 0.03 mL/(cm²·min·bar).

### Example 2

The same procedure as described in Example 1 was followed with the exception that the precipitation bath was conditioned to 60° C. The hollow-fiber membranes obtained had a physical inner diameter of about 250 micrometers, a wall thickness of about 67 micrometers, a zone with minimum pore size located adjoining the inner surface, and a zone with maximum pore size located approximately in the middle of the membrane. In addition, pores on the outer surface were larger in size than the pores on the inner surface. The transmembrane flow (TMF) was measured to be 0.03 mL/(cm²·min·bar).

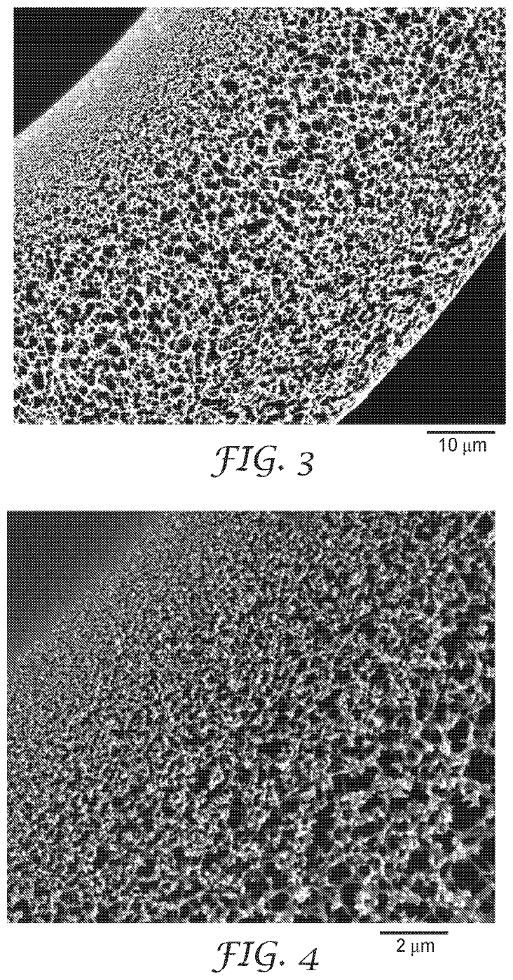

Scanning electron microscope (SEM) images of cross-sections of the hollow-fiber membrane are shown in FIG. 3 and FIG. 4.

### Example 3

The same procedure as described in Example 1 was followed except that the rotational speed of the polymeric blend pump was lowered. The hollow-fiber membranes obtained had a physical inner diameter of about 250 micrometers, a wall thickness of about 30 micrometers, a zone with minimum pore size located adjoining the inner surface, and a zone with maximum pore size located approximately in the middle of the membrane. In addition, pores on the outer surface are larger in size than the pores on the inner surface. The transmembrane flow (TMF) was measured to be 0.10 mL/(cm²·min·bar).

### Example 4

The same procedure as described in Example 1 was followed with the exception that the precipitation bath was conditioned to 50° C. and the rotational speed of the polymeric blend pump was lowered. The hollow-fiber membranes obtained had a physical inner diameter of about 250 micrometers, a wall thickness of about 30 micrometers, a zone with minimum pore size located adjoining the inner surface, and a gradient of increasing pore size towards the outer surface. The maximum pores were located adjoining (<1 micrometer in distance) the outer surface. The transmembrane flow (TMF) was measured to be 0.18 mL/(cm²·min·bar).

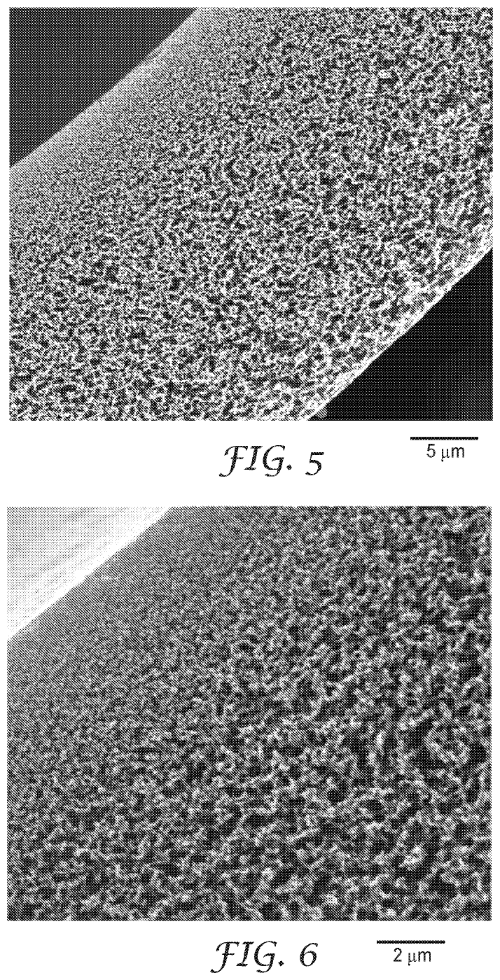

Scanning electron microscope (SEM) images of cross-sections of the hollow-fiber membrane are shown in FIG. 5 and FIG. 6.

### Example 5

A spinning solution was prepared by vigorously mixing 21 wt. % polyethersulfone, 9 wt. % poly(2-ethyl-2-oxazoline), 36 wt. % N-methylpyrrolidone, 32 wt. % poly(ethylene glycol) 200 (PEG200), and 2% water at a temperature of about 55° C. The resulting spinning solution was cooled down to about 50° C., filtered, and degassed. A temperature controlled spinneret (35° C.) having an outer diameter for dope of 0.41 mm, a needle outer diameter of 0.3 mm and a spinneret needle inner diameter of 0.15 mm was used. The spinneret was fixed at a distance of 25 cm above the precipitation bath.

Using the above-mentioned spinning solution and a mixture of NMP:polyethylene glycol:water ( 50:30:20) as the bore liquid in the spinneret needle of the spinneret, a hollow fiber was generated. Next, the hollow fiber was transferred into the water-containing precipitation bath heated to about 60° C., thereby fixing the membrane structure with the minimum sized pores at the outer surface region and the maximum sized pores at the inner (lumen) surface of the membrane. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at about 90° C. for about one hour. The hollow-fiber membranes obtained had a physical inner diameter of about 300 micrometers and a wall thickness of about 50 micrometers.

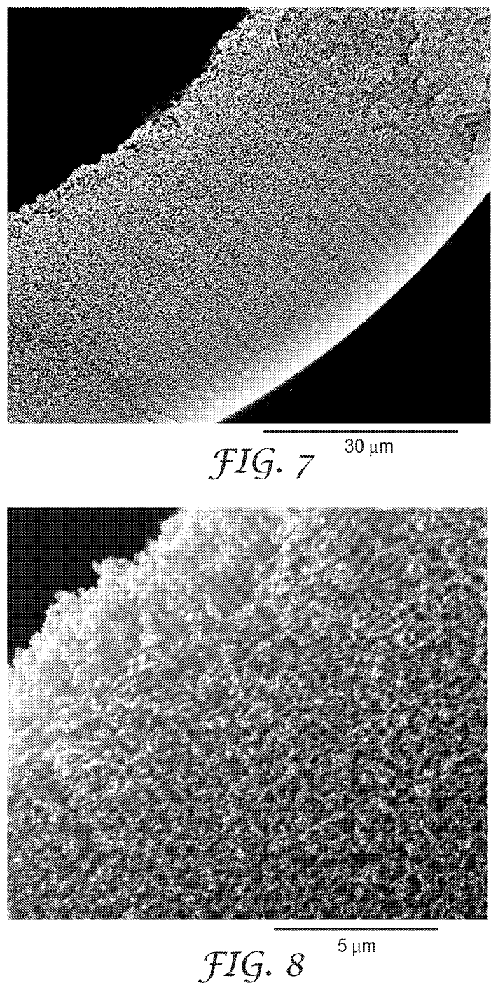

Scanning electron microscope (SEM) images of cross-sections of the hollow-fiber membrane are shown in FIG. 7 and FIG. 8. In FIG. 7, a zone with minimum pore size is located adjoining the outer surface and a zone with maximum pore size is located adjoining the inner surface. The image of FIG. 8 shows the interconnected, tortuous structure of the membrane wall.

The transmembrane flow (TMF) was measured to be 0.11 mL/(cm²·min·bar). An LRV of 1.5 for Phi X174 phage was measured when the membrane was evaluated according to Methods E and F.

### Example 6

A spinning solution was prepared by vigorously mixing 19 wt. % polyethersulfone, 13 wt. % poly(2-ethyl-2-oxazoline), 63 wt. % N-methylpyrrolidone, and 5% water at a temperature of about 55° C. The resulting spinning solution was cooled down to about 50° C., filtered, and then degassed. A temperature controlled spinneret (35° C.) and having an outer diameter for dope of 0.41 mm, a needle outer diameter of 0.3 mm, and a spinneret needle inner diameter of 0.15 mm was used. The spinneret was fixed at a distance of 60 cm above the precipitation bath.

Using the above-mentioned spinning solution and a mixture of NMP:water (53:47) as the bore liquid in the spinneret needle of the spinneret, a hollow fiber was generated. This hollow fiber was transferred through a climate-controlled zone conditioned to a temperature of 25-75° C. and 20-95% relative humidity. Next, the hollow fiber was transferred into the water-containing precipitation bath heated to about 60° C., thereby fixing the membrane structure to have maximum sized pores at the outer surface region and minimum pores on the inner (lumen) surface of the membrane. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at about 90° C. for about one hour. The hollow-fiber membranes obtained had a physical inner diameter of about 250 micrometers, a wall thickness of about 70 micrometers, a zone with minimum pore size located adjoining the inner surface, and a gradient of increasing pore size towards the outer surface. The maximum size pores were located adjoining (<1 micrometer in distance) the outer surface.

The transmembrane flow (TMF) was measured to be 0.8 mL/(cm²·min·bar). The BSA throughput was measured to be 73 kg/(m²·hr).

### Example 7

A spinning solution was prepared by vigorously mixing 19 wt. % polyethersulfone, 13 wt. % poly(2-ethyl-2-oxazoline), 63 wt. % N-methylpyrrolidone, and 5 wt. % water at a temperature of about 55° C. The resulting spinning solution was cooled down to about 50° C., filtered, and degassed. A temperature controlled spinneret (35° C.) having an outer diameter for dope of 0.41 mm, a needle outer diameter of 0.3 mm and a spinneret needle inner diameter of 0.15 mm was used. This spinneret was fixed at a distance of 25 cm above the precipitation bath.

Using the above-mentioned spinning solution and a mixture of NMP:water (53:47) as the bore liquid in the spinneret needle of the spinneret, a hollow fiber was generated. Next, the hollow fiber was transferred into the water-containing precipitation bath heated to about 71° C., thereby fixing the membrane structure with the minimum sized pores at the outer surface region and the maximum sized pores at the inner (lumen) surface of the membrane. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at about 90° C. for about one hour. The hollow-fiber membranes obtained had a physical inner diameter of about 250 micrometers, a wall thickness of about 70 micrometers, a zone with minimum pore size located adjoining the outer surface, and a zone with maximum pore size located adjoining the inner surface of the hollow fiber membranes.

The transmembrane flow (TMF) was measured to be 0.8 mL/(cm²·min·bar). The BSA throughput was measured to be 230 kg/(m²·hr).

### Example 8

A spinning solution was prepared by vigorously mixing 19 wt. % polyethersulfone, 6.5 wt. % poly(2-ethyl-2-oxazoline), 6.5 wt. % PEG1500, 63 wt. % N-methylpyrrolidone, and 5 wt. % water at a temperature of about 55° C. The resulting spinning solution was cooled down to about 50° C., filtered, and then degassed. A temperature controlled spinneret (35° C.) and having an outer diameter for dope of 0.41 mm, a needle outer diameter of 0.3 mm and a spinneret needle inner diameter of 0.15 mm was used. This spinneret was fixed at a distance of 25 cm above the precipitation bath.

Using the above-mentioned spinning solution and a mixture of NMP:water (53:47) as the bore liquid in the spinneret needle of the spinneret, a hollow fiber was generated. Next, the hollow fiber was transferred into the water-containing precipitation bath heated to about 71° C., thereby fixing the membrane structure with the minimum sized pores at the outer surface region and the maximum sized pores at the inner (lumen) surface of the membrane. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at a temperature of about 90° C. for about one hour. The hollow-fiber membranes obtained had a physical inner diameter of about 250 micrometers, a wall thickness of about 70 micrometers, a zone with minimum pore size located adjoining the outer surface, and a zone with maximum pore size located adjoining the inner surface of the hollow fiber membranes.

The transmembrane flow (TMF) of the membrane was measured to be 0.8 mL/(cm²·min·bar). The BSA throughput was measured to be 1046 kg/(m²·hr).

### Example 9

A spinning solution was prepared by vigorously mixing 24 wt. % polyethersulfone, 9 wt. % poly(2-ethyl-2-oxazoline), 34 wt. % N-methylpyrrolidone, 31 wt. % poly(ethylene glycol) 200 (PEG200), and 2 wt. % water at a temperature of about 55° C. The resulting spinning solution was cooled down to about 50° C., filtered, and degassed. A temperature controlled spinneret (35° C.) having an outer diameter for dope of 0.41 mm, a needle outer diameter of 0.3 mm and a spinneret needle inner diameter of 0.15 mm was used. The spinneret was fixed at a distance of 25 cm above the precipitation bath.

Using the above-mentioned spinning solution and a mixture of NMP:polyethylene glycol(PEG200):water (50:45:5) as the bore liquid in the spinneret needle of the spinneret, a hollow fiber was generated. Next, the hollow fiber was transferred into the water-containing precipitation bath heated to about 60° C. Directly after this coagulation and fixation step, the wet hollow-fiber membranes were wound on a wheel and then assembled into a hollow-fiber membrane bundle having a length of about 30 cm and comprising about 1200 individual hollow-fiber membranes. The hollow-fiber membranes were extracted with hot water (about 90° C.) for about one hour and then dried with air at about 90° C. for about one hour. The hollow-fiber membranes obtained had a physical inner diameter of about 217 micrometers and a wall thickness of about 61 micrometers. The transmembrane flow (TMF) was measured to be 0.69 mL/(cm²·min·bar).

The cross-section of the membrane wall was examined using a SEM (8,000× magnification). The pore size of the membrane at the inner surface facing the lumen was about 0.2-5 micrometers. The pore size progressively decreased in the direction from the inner membrane surface to the outer membrane surface for a distance of about 54 micrometers (89%) across the membrane wall (the measurement of pore size was made along a vector that defined the shortest cross-section distance from the inner membrane surface to the outer surface of the membrane). The pore size then transitioned to progressively increase in size (along the same vector direction) toward the outer surface with the pore size at the outer membrane surface being about 0.2-3 micrometers. The pore size at the location in the membrane wall where the pore size transitioned from increasing to decreasing was about 0.04 micrometers.

### Example 10

Hollow-fiber membranes were prepared according to the general procedure described in Example 9 with the exception that the components of the spinning solution were added in different weight percentages. The spinning solution was prepared by vigorously mixing 25.5 wt. % polyethersulfone, 9 wt. % poly(2-ethyl-2-oxazoline), 33.2 wt. % N-methylpyrrolidone, 30.3 wt. % poly(ethylene glycol) 200 (PEG200), and 2 wt. % water. The hollow-fiber membranes obtained had a physical inner diameter of about 216 micrometers and a wall thickness of about 57 micrometers. The transmembrane flow (TMF) was measured to be 0.97 mL/(cm²·min·bar).

The cross-section of the membrane wall was examined using a SEM (8,000× magnification). The pore size of the membrane at the inner surface facing the lumen was about 0.3-3 micrometers. The pore size progressively decreased in the direction from the inner membrane surface to the outer membrane surface for a distance of about 48 micrometers (84%) across the membrane wall (the measurement of pore size was made along a vector that defined the shortest cross-section distance from the inner membrane surface to the outer surface of the membrane). The pore size then transitioned to progressively increase in size (along the same vector direction) toward the outer surface with the pore size at the outer membrane surface being about 0.4-1.3 micrometers. The pore size at the location in the membrane wall where the pore size transitioned from increasing to decreasing was about 0.045-0.05 micrometers.

### Example 11

Hollow-fiber membranes were prepared according to the general procedure described in Example 9 with the exception that the components of the spinning solution were added in different weight percentages. The spinning solution was prepared by vigorously mixing 26.5 wt. % polyethersulfone, 9 wt. % poly(2-ethyl-2-oxazoline), 32.7 wt. % N-methylpyrrolidone, 29.8 wt. % poly(ethylene glycol) 200 (PEG200), and 2 wt. % water. The hollow-fiber membranes obtained had a physical inner diameter of about 203 micrometers and a wall thickness of about 53 micrometers. The transmembrane flow (TMF) was measured to be 0.45 mL/(cm²·min·bar).

The cross-section of the membrane wall was examined using a SEM (8,000× magnification). The pore size of the membrane at the inner surface facing the lumen was about 6.5-0.3 micrometers. The pore size progressively decreased in the direction from the inner membrane surface to the outer membrane surface for a distance of about 50 micrometers (94%) across the membrane wall (the measurement of pore size was made along a vector that defined the shortest cross-section distance from the inner membrane surface to the outer surface of the membrane). The pore size then transitioned to progressively increase in size (along the same vector direction) toward the outer surface with the pore size at the outer membrane surface being about 0.05-0.3 micrometers. The pore size at the location in the membrane wall where the pore size transitioned from increasing to decreasing was less than 0.03 micrometers.

### Example 12

Hollow-fiber membrane test modules were prepared and tested according to the following procedure. Polycarbonate tubes with lengths of 13 mm and inner diameters of 4 mm were used. A single hole was drilled in the side of each tube. An open-bore connector was attached to the hole using a uv/visible light cured adhesive to form a side port. About 10-15 hollow-fibers prepared according to Example 10 were placed in each tube. The inserted hollow-fibers were cut with a razor blade to provide an overhang of hollow-fibers of about 15 mm at each end of the tube. The overhanging hollow-fibers were sealed with wax and then potted in the tube using a polyurethane resin. After curing for 24 hours, the protruding ends were removed using a razor blade. The openings of the membranes were inspected using a microscope and only tubes in which all the hollow-fiber membranes had open and unobstructed lumen portions were used. The total inner hollow-fiber surface area (i.e., total lumen surface area) was about 1 cm². Test modules were sterilized using gamma irradiation (25-45 kGy) prior to being challenged with an AAV solution.

The membrane test module was connected to a three-way valve located at the bottom of a vertically mounted pressure pot. The pressure pot was initially filled with ultrapure water (obtained from a MILLI-Q water purification system, MilliporeSigma). The three-way valve between the pressure pot and test module was opened allowing the water to flow into the lumens of the hollow-fibers and out the opposite end of the module. When the lumens of the hollow-fibers were filled with water, the end of the test module was capped. The pressure was gradually increased to 30 psi. The side port on the test module was used to allow the filtrate to exit the module into a first collection vessel. The water was filtered through the hollow-fiber module for a minimum of 10 minutes at 30 psi. The three-way valve at the bottom of the pressure pot was then closed and any remaining water in the pressure pot was removed.

The pressure pot was depressurized and filled with an AAV serotype 2 (AAV2) (Vector BioLabs, Malvern, PA) solution at a concentration of about 1×10⁹ capsids/mL in phosphate buffer saline. The pressure pot was then sealed, pressurized to 30 psi, and the three-way valve was opened. The filtrate was collected in a tared, second collection vessel that was placed on a digital balance. A minimum of 100 L/m² of AAV2 solution was filtered through the membrane test module. The AAV2 concentration in the filtrate and the feed was measured using a PROGEN Xpress AAV2 ELISA kit (PROGEN, Wayne, PA). The percent yield of AAV2 was calculated by dividing the concentration of the AAV2 in the filtrate by the concentration of AAV2 in the feed and multiplying by 100. The calculated percent yield of AAV2 was 100%.

### Example 13

Bacteriophage T7 was used to simulate a viral contaminant larger in size than AAV and to demonstrate the ability of the membrane to remove a large viral contaminant T7 phage were spiked into phosphate buffer (pH 7.4, 4 mS/cm) at a concentration of 1×10⁷ pfu/mL to prepare the feed solution. The filtration of the phage containing feed solution was conducted using a constant flow rate during the filtration.

Hollow-fiber membrane test modules (polycarbonate tubes 13 mm in length, 4 mm inner diameter, with 10-15 fibers each for a total filtration area of 5-6 cm²) were prepared as described in Method E (using hollow-fibers prepared in Example 10) and sterilized using gamma irradiation (25-45 kGy). The membrane test module was mounted vertically. A pressure gauge was placed in line at the inlet of the vertically mounted module. A peristaltic pump and MASTERFLEX tubing (size 14, Cole-Parmer) were used to pump the challenge solution from container into the lumens of the hollow fibers. The pump was set at a flow rate that provided a transmembrane pressure of 20-30 psi. Ultrapure water (obtained from a MILLI-Q water purification system, MilliporeSigma) was initially pumped into the module while the opposite end of the module was uncapped, allowing the water to completely fill the membrane lumens. Once the lumens were filled with water, the module was capped and the module was operated in dead-end filtration mode. The side port on the test module was used to allow the filtrate to exit the module. The filtrate was collected in a tared beaker placed on a digital balance. Ultrapure water was filtered through the test module for a minimum of 10 minutes and the transmembrane pressure monitored. After the initial water flush, the inlet feed was switched to the T7 phage feed solution. The initial filtrate that would have represented the dead volume of the system was collected and discarded. Water was drained from the inlet line. The T7 phage feed solution was pumped at the same flow rate as used with the water, providing a transmembrane pressure of 20-30 psi. A minimum of 100 L/m² of the feed solution was used to challenge the membrane. The filtrate was collected in a tared, sterile container placed on a digital balance. The transmembrane pressure was monitored throughout the experiment. The T7 phage concentration of the filtrate and the corresponding LRV value were determined according to Method H. An LRV of >6 was measured for the T7 phage.

All references and publications cited herein are expressly incorporated herein by reference in their entirety into this disclosure. Illustrative embodiments of this invention are discussed and reference has been made to possible variations within the scope of this invention. For example, features depicted in connection with one illustrative embodiment may be used in connection with other embodiments of the invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A hollow-fiber membrane;
the hollow-fiber membrane made from a polymeric blend comprising an aromatic sulfone polymer and a polyoxazoline;
wherein the hollow-fiber membrane comprises an inner surface facing towards its lumen, an outer surface facing outwards and an intermediate wall having a wall thickness;
wherein the hollow-fiber membrane is an integrally asymmetric, permeable hollow-fiber membrane.

2. The hollow-fiber membrane of claim 1, wherein the aromatic sulfone polymer comprises a polysulfone or polyethersulfone.

3. The hollow-fiber membrane of claim 1, wherein the polyoxazoline is poly(2-ethyl-2-oxazoline) (PEtOx).

4. The hollow-fiber membrane of claim 1, wherein the polyoxazoline has a molecular weight of from about 25 kg/mol to about 500 kg/mol.

5. The hollow-fiber membrane of claim 1, wherein a zone with minimum pore size adjoins the inner surface.

6. The hollow-fiber membrane of claim 5, wherein the zone with maximum pore size adjoins the outer surface.

7. The hollow-fiber membrane of claim 5, wherein the zone with minimum pore size adjoins to the outer surface.

8. The hollow-fiber membrane of claim 7, wherein the zone with maximum pore size adjoins the inner surface.

9. The hollow-fiber membrane of claim 5, wherein the size of the pores in the zone with minimum pore size is in the range of from 10 nm to 100 nm.

10. The hollow-fiber membrane of claim 5, wherein the size of the pores in the zone with minimum pore size is in the range of from 30 nm to 60 nm.

11. The hollow-fiber membrane of claim 1, wherein the polymeric blend further comprises a hydrophilic polymer and a solvent.

12. The hollow-fiber membrane of claim 11, wherein the hydrophilic polymer is selected from polyvinylpyrrolidone, polyethylene glycol, Glycerol, polyvinyl alcohol, polyglycol monoester, polysorbitate, carboxymethylcellulose, polyacrylic acid, polyacrylate, or a modification or a copolymer of these polymers.

13. The hollow-fiber membrane of claim 11, wherein the solvent is a polar, aprotic solvent or a protic solvent.

14. The hollow-fiber membrane of claim 11, wherein the solvent comprises N-methylpyrrolidone.

15. The hollow-fiber membrane of claim 1, wherein the polymeric blend comprising more than 7 wt. % or less than 3 wt. % of polyvinylpyrrolidone.

16. The hollow-fiber membrane of claim 1, wherein the polymeric blend does not comprise polyvinylpyrrolidone.

17. The hollow-fiber membrane of claim 1, wherein the hollow-fiber membrane comprises tortuous structures extending from the inner surface toward to the outer surface.

18. A method, comprising:
providing a spinning solution comprising an aromatic sulfone polymer and polyoxazoline, and a bore liquid comprising water, a solvent and a non-solvent; and
spinning a hollow-fiber with a spinneret outer diameter for dope in the range of from 300 to 1000 μm, a spinneret needle outer diameter in the range of from 200 to 1000 μm and a spinneret needle inner diameter in the range of from 100 to 1000 μm.

19-21. (canceled)

22. A use of the hollow-fiber membrane of claim 1 for filtration of liquids.

23. The use of claim 22, wherein the liquids comprise a biological product selected from adeno-associated virus (AAV) capsids, viruses, virus like particles.

24-27. (canceled)
